(19) United States
(12) Patent Application Publication
(10) Pub. No.: US 2002/0155131 A1
(43) Pub. Date: Oct. 24, 2002

(54) Recombinant equine herpesvirus type 1 (EHV-1) comprising a dysfunctional gene 71 region and use thereof as a vaccine

(75) Inventors: Susanne Moira Brown, Glasgow (GB); Yi Sun, Memphis, TN (US); Hugh John Field, Cambridge (GB)

Correspondence Address: FOLEY HOAG LLP, PATENT GROUP, 155 SEAPORT BOULEVARD, BOSTON, MA, 02110, US

(73) Assignee: University Court of The University of Glasgow

(21) Appl. No.: 10/033,538

(22) Filed: Oct. 19, 2001

Related U.S. Application Data
(63) Continuation of application No. 09/319,614, filed on Nov. 23, 1999, now abandoned.

(30) Foreign Application Priority Data
Dec. 13, 1996 (GB) GB9625968.4

(51) Int. Cl. A61K 39/12

(52) U.S. Cl. 424/205.1

(57) ABSTRACT

Vaccine formulation comprising EHV-1 gene 71 dysfunctional mutant and uses thereof.

The present invention relates to a viral vaccine containing an attenuated EHV-1 virus comprising a gene deletion in the genome thereof, uses thereof and methods of treating EHV-1 related disease. In particular, the invention relates to a viral vaccine composition for use against Equine herpesvirus type 1 (EHV-1).

EHV-1 is a member of the subfamily alphaherpesvirinae and is a significant viral pathogen of horses. Clinical problems caused by EHV-1 include respiratory disease, abortion and neurological disorders (Bryans J. T., and Allen, G. P., Kluwer Academic Publishers, Norwell Mass., 1989). As such, EHV-1 is responsible for significant economic losses within the equine industry.

The EHV-1 genome is a linear double-stranded DNA molecule of approximately 150 kbp in size which can be divided into two covalently linked components: the long and short regions. The long region consists of an unique sequence (U_{L}) flanked by a small inverted repeat (IR_{L} and TR_{L}). The short region comprises an unique sequence (U_{S}) flanked by a large inverted repeat (IR_{S} and TR_{S}).

EHV-1 occurs as pathogenic and non-pathogenic strains and recently, the complete DNA sequence of a pathogenic strain, Ab4, has been determined and the sequence has been deposited with the GenBank Library under Accession No. M 86664 (Telford, E. A. R. et al., Virology 189, pp. 304-316 (1990)).

The genome is 150,223 bp in size and contains 81 open reading frames predicted to encode polypeptides. The sizes of its components are U_{L}, 112,870 bp; TR_{L}/IR_{L}, 32 bp; U_{S}, 11,861 bp; and IR_{S}/TR_{S}, 12,714 bp. Interestingly, there are five genes, 1, 2, 67, 71, and 75, which have no homologues in any of the herpesviruses sequenced to date; i.e. they are unique to EHV-1.

Each of the genes 1, 2, 67, 71 and 75 is believed to encode a protein, however, the function of the individual proteins is unclear. Recently it has been demonstrated that the EHV-1 gene 71 product is involved in adsorption/penetration of virus and egress of virus from infected cell nuclei (Sun Y. et al., Journal of General Virology 77 pp. 493-500 (1996)).

The prior art does not teach or suggest the use of EHV-1 gene 71 deletion mutants comprising a dysfunctional gene 71 region in the manufacture and use of vaccines against EHV-1 related disease.

Control by vaccination of EHV-1 infection has been a long-sought goal. Current EHV-1 vaccines comprise chemically inactivated virus vaccines and modified live virus vaccines. Inactivated vaccines generally induce a low level of immunity and require additional immunisations and are expensive to produce. The use of such vaccines carries with it the risk that some infectious viral particles may survive the inactivation process and cause disease after administration to the animal.

In general, attenuated live virus vaccines are preferred because they evoke a longer-lasting immune response (often both humoral and cellular) and are easier to produce. Live attenuated EHV-1 vaccines are available which are based on live EHV-1 virus attenuated by serial passage of virulent strains in tissue culture. However, serial passaging of virulent strains can give rise to uncontrolled mutations of the viral genome, resulting in a population of virus particles heterogeneous in their virulence and immunising properties. It is also known that such EHV-1 attenuated live virus vaccines can revert to virulence resulting in disease of the inoculated animals and the possible spread of pathogen to other animals.

The present inventors have now identified a suitable strain of live EHV-1 mutant virus comprising a dysfunctional region of the EHV-1 genome located within the short unique region thereof, which mutant may be used in a live EHV-1 vaccine formulation. Specifically, the inventors have found that EHV-1 mutants dysfunctional for production of a protein encoded by gene 71 can be used in a live EHV-1 vaccine formulation. Such mutants are shown to be substantially less virulent than wild type EHV-1 viruses. Furthermore, gene 71 has been found to be non-essential for EHV-1 growth in cell culture (Sun Y. and Brown S. M., Virology 199 pp. 448-452 (1994)). The inventors have also found that EHV-1 viruses comprising dysfunctional gene 71 regions of their genome are immunogenic. Such viruses are indicated for use as components in vaccine formulations or therapeutic compositions against EHV-1 infection. Accordingly, it is with EHV-1 viruses comprising a dysfunctional region located in the gene 71 protein coding region, and in particular between nucleotides 129,096 and 131,489 of the native genome which the present invention is concerned.

### STATEMENT OF INVENTION

A first aspect of the present invention provides a vaccine formulation comprising a live recombinant EHV-1 virus modified so as to contain a dysfunctional gene 71 region located within the U_{S} region of the virus genome and a pharmaceutically acceptable carrier.

A “dysfunctional gene 71 region” is one which is substantially incapable of coding for the native polypeptide or a functional equivalent. Thus, a “dysfunctional gene 71 region” means that the gene 71 region has been modified by deletion, insertion or substitution (or other change in the DNA sequence such as by rearrangement) such that the gene 71 region does not express a native EHV-1 gene 71 polypeptide or a functionally equivalent product thereof. It is known that EHV-1 gene 71 encodes a 797 amino acid polypeptide and that the peptide is an O-linked 192 kDa glycoprotein (Sun, Y. et al., Journal of General Virology 75, pp. 3117-3126 (1994)). Thus, vaccine formulations comprising modified EHV-1 viruses of the invention may include viruses modified in one or more ways via recombinant DNA technology. Examples of the types of modifications which may be made include:

(i) A deletion of the entire gene 71 from the genome of an EHV-1 wild type virus. For example, a deletion of the nucleotide sequence from the wild type EHV-1 genome between about nucleotide 129,096 to about nucleotide 131,489.

(ii) A deletion of a portion of gene 71 from the genome of an EHV-1 wild type virus. A “portion of the gene 71” means a deletion which is sufficient to render any polypeptide encoded by the gene 71 deletion mutant and expressed thereby substantially incapable of a physiological activity similar to that of the native polypeptide produced by wild type EHV-1. The deletion may be between 50% and 100% of the nucleotide sequence located between about nucleotides 129,096 and 131,489 of the wild type EHV-1 genome. The deletion may be from 70% to 100% of the gene 71 nucleotide sequence, or the deletion may be from about 70% to 90% of the gene 71 nucleotide sequence, for example, about 80% of the gene 71 nucleotide sequence.

(iii) The deletion of the or a portion of gene 71 as described in (i) and (ii) above will leave a “gap” in the EHV-1 genome corresponding to the gene 71 open reading frame (ORF) or a portion thereof. A suitable gene or genes may be inserted into the “gap” such as a marker gene. Suitable marker genes include but are not restricted to enzyme marker genes, for example the lac-Z gene from E.coli, antibiotic marker genes such as hygromycin, neomycin and the like. Such marker genes are commonly employed in the art. Generally, marker genes, if any, which may be employed in a gene 71 deletion mutant of the invention should be such so as to not cause substantial deleterious or long lasting side-effects to a recipient animal.

In a preferment, the “gap” made by the deletion of the or a portion of the gene 71 from a wild type EHV-1 virus is not filled with a gene insert, the cut ends of the two pieces of the genome being ligated together using conventional recombinant DNA technology. The skilled addressee will appreciate that the term “deletion mutant” encompasses those situations wherein the “gap” left by the partial or total deletion of gene 71 may be filled with a gene insert, for example a marker gene or nonsense nucleotide sequence (i.e. a sequence incapable of giving rise to a protein or polypeptide product) or those situations wherein the gap is not filled by a heterologous or other nucleotide sequence. In such a case, the appropriate free ends of the two pieces of the genome are ligated together.

(iv) The deletion within the gene 71 region may comprise a deletion of a small number of nucleotides, for example 1, 2 or more nucleotides. Such deletions can be achieved using recombinant DNA technology. Thus, the translational ORF can be altered resulting in the production of a protein which lacks the physiological function of the gene 71 native polypeptide. The skilled addressee will also appreciate that such deletions in the translational ORF of gene 71 may also give rise to a dysfunctional gene 71 which is incapable of coding for a whole polypeptide, truncated peptide or even any peptide. Such proteins, if produced, generally lack the physiological functionality of the protein product of a normal gene 71 ORF.

(v) Nucleotide insertions can also be made in the EHV-1 gene 71 region using recombinant DNA technology which gives rise to dysfunctional gene 71 polypeptides substantially incapable of functional activity. For example, stop codons may be inserted into the gene 71 region, resulting in the production of non-functional fragments of the polypeptide encoded by native gene 71.

The skilled addressee will appreciate that such nucleotide insertions can be of any length from 1 or more nucleotides to a number of nucleotides making up, for example, nonsense nucleotide sequences which can have the effect of altering the translational ORF resulting in the non-production of a polypeptide or indeed, the production of a protein lacking the physiological function of the gene 71 native polypeptide. The skilled addressee will also appreciate that such insertions in the translational ORF of gene 71 may also give rise to a dysfunctional gene 71 which is incapable of coding for a whole polypeptide, truncated peptide or even any peptide. Such proteins, if produced, generally lack physiological functionality.

Naturally, the skilled addressee will appreciate that gene 71 deletions and insertions from non-wild type EHV-1 viruses as outlined above are encompassed by the present invention.

In a preferment there is provided a vaccine formulation comprising a live recombinant attenuated immunogenic EHV-1 gene 71 deletion mutant virus and a pharmaceutically acceptable carrier.

In a second aspect of the invention there is provided a live, recombinant EHV-1 comprising a dysfunctional gene 71 region for use as a vaccinating agent or in a vaccine formulation. Preferably, there is provided a live, recombinant, attenuated immunogenic EHV-1 gene 71 deletion mutant virus for use as a vaccinating agent or in a vaccine formulation.

The live, recombinant EHV-1 may optionally include an inserted gene positioned at the gene 71 locus in lieu of a substantial portion of gene 71 or the whole of gene 71.

Generally, the vaccine or vaccine formulation is not used on non-pregnant animals because it can give rise to abortigenesis.

In a third aspect of the invention there is provided the use of a live, recombinant EHV-1 virus for producing antibodies or cell mediated immunity to EHV-1 which comprises a dysfunctional gene 71 region located within the U_{S} region of the virus genome for the manufacture of an EHV-1 vaccine for the prophylaxis and/or treatment of EHV-1 infection. Preferably, there is provided use of a live, recombinant, attenuated immunogenic EHV-1 gene 71 deletion mutant virus for the manufacture of an EHV-1 vaccine for the prophylaxis and/or treatment of EHV-1 infection. Most preferably, the use is in horses.

In a fourth aspect of the invention there is provided a method of treating animals which comprises administering thereto a vaccine composition comprising a live, recombinant EHV-1 virus modified so as to contain a dysfunctional gene 71 region located within the U_{S} region of the virus genome to animals in need thereof. Preferably, the animals are horses. Preferably still, the method of treating animals comprises administering a vaccine composition comprising a recombinant, live, attenuated, immunogenic EHV-1 gene 71 deletion mutant virus to animals in need thereof. Naturally, the vaccine formulation may be formulated for administration by oral dosage (e.g. as an enteric coated tablet), by parenteral injection or otherwise.

The invention also provides a process for preparing a live modified EHV-1 virus vaccine, which process comprises admixing a virus according to the invention with a suitable carrier or adjuvant.

For the preparation of a live attenuated vaccine, standard methodology may be used.

The mode of administration of the vaccine of the invention may be by any suitable route which delivers an immunoprotective amount of the virus of the invention to the subject. However, the vaccine is preferably administered parenterally via the intramuscular or deep subcutaneous routes. Other modes of administration may also be employed, where desired, such as oral administration or via other parenteral routes, i.e., intradermally, intranasally, or intravenously.

Generally, the vaccine will usually be presented as a pharmaceutical formulation including a carrier or excipient, for example an injectable carrier such as saline or apyrogenic water. The formulation may be prepared by conventional means.

The appropriate immunoprotective and non-toxic dose of such a vaccine can be determined readily by those skilled in the art, i.e., the appropriate immunoprotective and non-toxic amount of the virus contained in the vaccine of this invention may be in the range of the effective amounts of antigen in conventional whole virus vaccines. It will be understood, however, that the specific dose level for any particular recipient animal will depend upon a variety of factors including age, general health, and sex; the time of administration; the route of administration; synergistic effects with any other drugs being administered; and the degree of protection being sought. Of course, the administration can be repeated at suitable intervals if necessary.

Embodiments of the invention will now be illustrated by way of the following Figures and Examples.

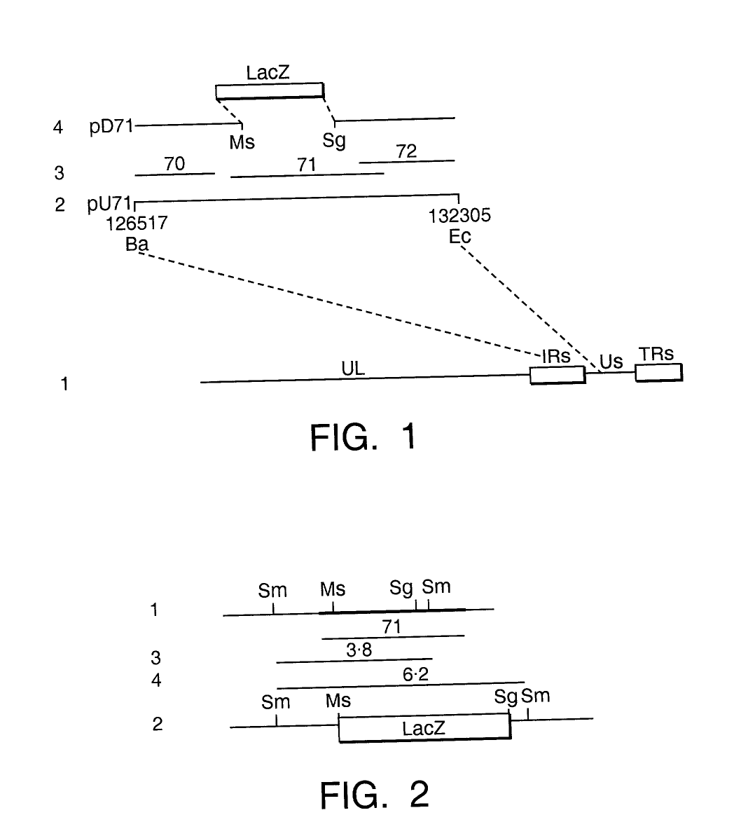

FIG. 1:

Schematic representation of the sequence arrangement of EHV-1 DNA and plasmids constructed for gene 71: deletion and substitution. Line 1, EHV-1 genome consisting of U_{L} and U_{S} and inverted repeat regions (IR_{S} and TR_{S}). Expanded cloned fragment: 5.8-kb BamHI/EcoRI fragment in pU71 (line 2). Line 3, location and direction of genes. Line 4, sequence arrangement of constructed deletion and substitution plasmid pD71 of gene 71 (line 4). Gaps flanked by solid lines represent deleted regions substituted by lacZ (solid boxes). Pertinent restriction sites: Ba, Ec, EcoRI; Ms, Sg and Bam HI.

FIG. 2:

Genome structure of the deletion and substitution mutant. Restriction enzyme sites within the region of the genome encompassing gene 71 are shown. The wild-type virus genome is represented by line 1, and the deletion and substitution by line 2. Relevant fragments generated following digestion with SmaI are shown. Fragment sizes are given in kb. Pertinent retriction sites Ms, Sm, Sq.

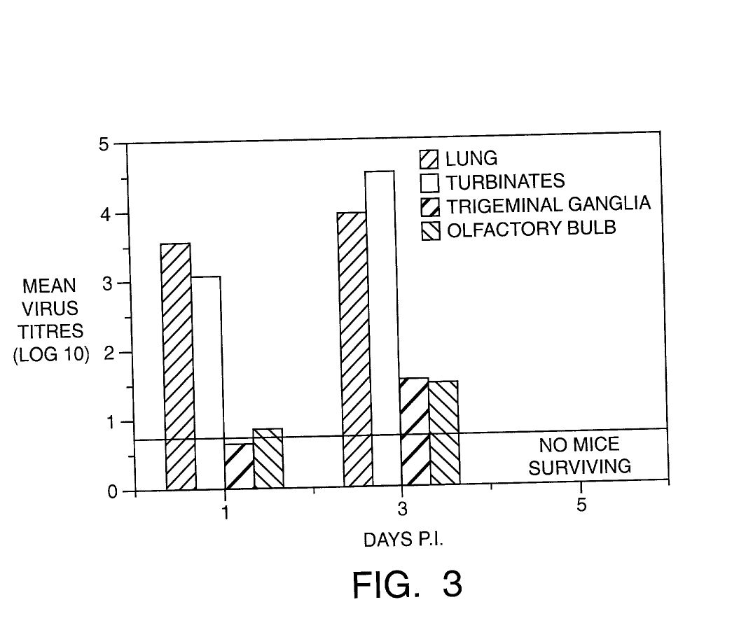

FIG. 3:

Virus titres for mice inoculated with Ab4p.

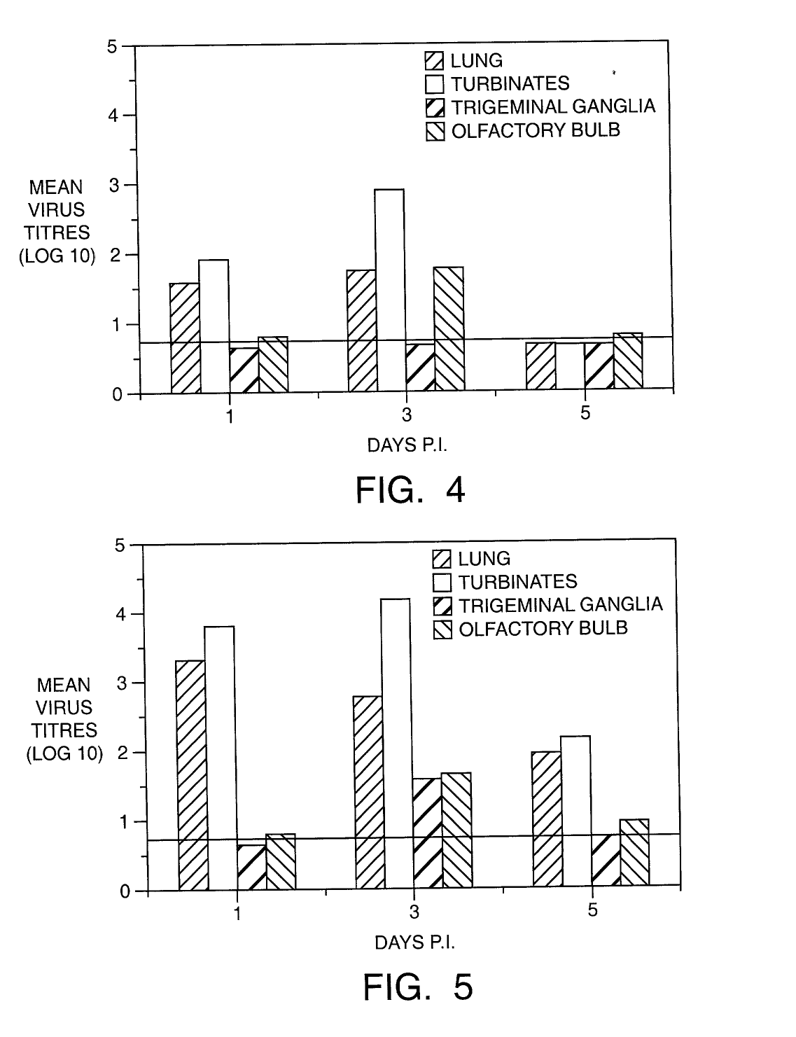

FIG. 4:

Virus titres for mice inoculated with ED 71.

FIG. 5:

Virus titres for mice inoculated with ED 71 revertant.

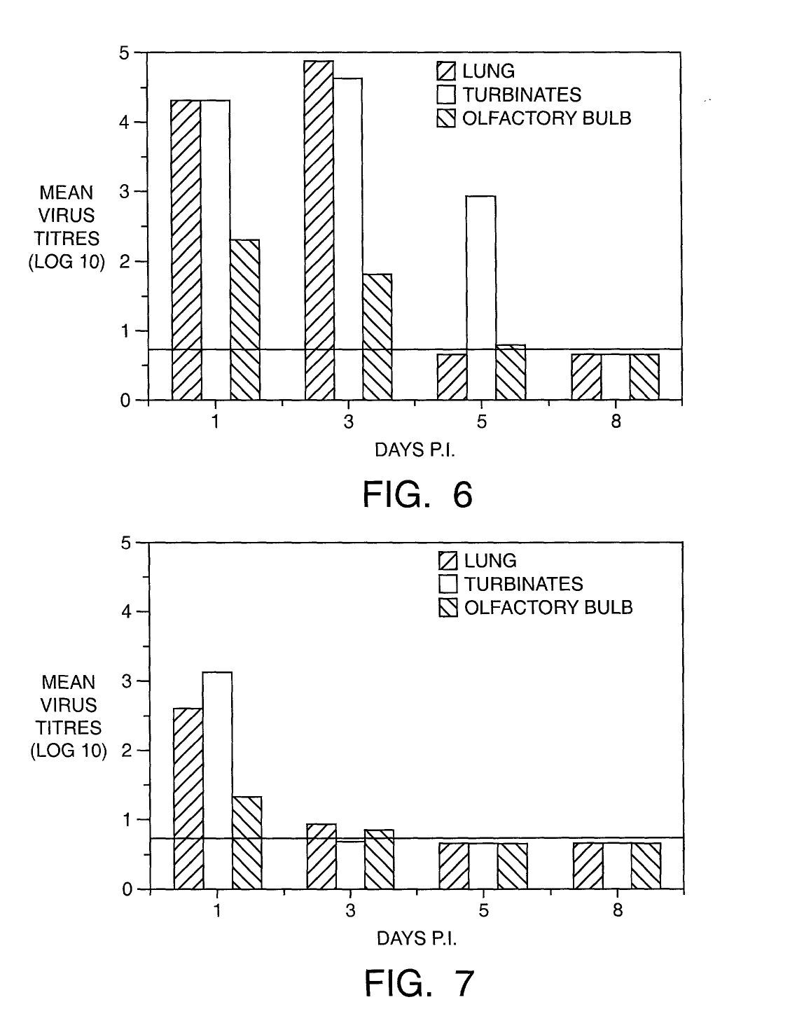

FIG. 6:

Mean virus titres observed in challenged mice previously immunised with RK cell lysate.

FIG. 7:

Mean virus titres observed in challenged mice previously immunised with Ab4p.

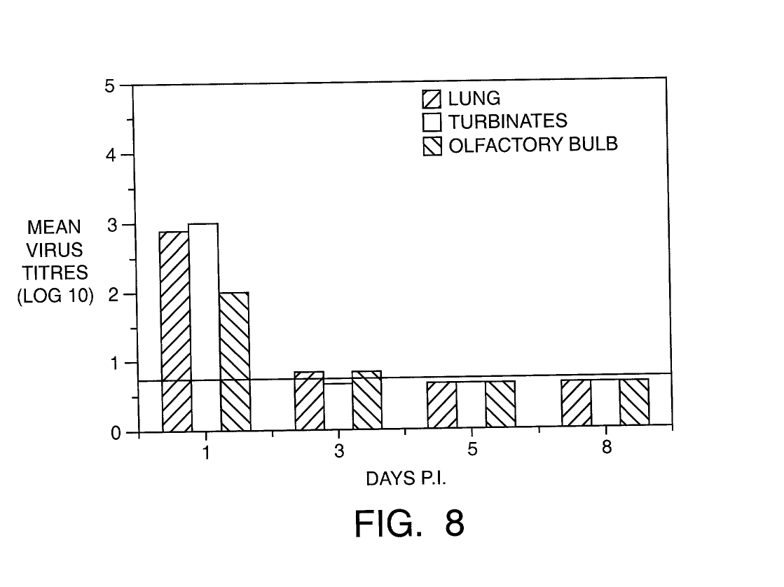

FIG. 8:

Mean virus titres observed in challenged mice previously immunised with ED 71.

Standard methods are as described in “Molecular Cloning-A Laboratory Manual”, Second Edition, Sambrook J. et al. Cold Spring Harbor Laboratory Press 1989.

### EXAMPLES SECTION 1

### Methods

Cells and Virus

Baby hamster kidney clone 13 (BHK-21/C13; Macpherson I & Stoker M. G. (1962) Virology 16 pp. 147-151) were grown as previously described (Brown et al., 1973 J. Gen. Virol. 18 pp. 32-346). EHV-1 strain Ab4 was used as the wild-type strain in this study. Stock preparation of virus at passage 13 was made by low multiplicity infection in equine dermal NBL-6 cells maintained in MEM with 1% fetal calf cerum. Mutant ED71 in which the gene 71 ORF was removed and replaced by the E.coli lacZ gene and the revertant Re71 in which the deletion in ED71 was restored have been previously described (Sun Y. and Brown S. M. (1994) Virology 199 pp. 448-452; Sun Y. et al. (1994) J. Gen. Virol. 75 pp. 3117-3126)

Purification and Quantification of Virions

The procedure used was essentially as described by Szilágyi J. F. and Cunningham C. (1991) J. Gen. Virol. 27 pp. 661-668 and Sun et al. (1994) supra. BHK-21/C13 monolayers in roller bottles were infected with virus at a multiplicity of infection (m.o.i.) of 0.01 or 5 p.f.u. per cell. At either 72 hours post-infection (p.i.) or 20 hours p.i. the supernatant was harvested and centrifuged at 2500 r.p.m. for 20 minutes to remove the cell debris. Supernatant virus was pelleted for 2 hours at 12000 r.p.m. and the pellet gently resuspended in 1 ml Eagle's medium without phenol red and laid onto a 5 to 15% Ficoll gradient before centrifuging at 12000 r.p.m. for 2 hours at 4° C. The virion band collected by side puncture was diluted and pelleted at 21000 r.p.m. for 2 hours at 4° C. The virion pellet was gently resuspended in 200 μl of Eagle's medium and stored at −70° C. Infectivity was determined by titration on BHK-21/C13 cells. The number of particles was determined by eletron microscopy. The specific infectivities (particle p.f.u. ratio) of the purified mutant and wild-type virus are presented in Table 1.

TABLE 1
The Specific Infectivity (particle/p.f.u. ratio) of
ED71, and Wild-Type Virus EHV-1).

| Virus | Particle/p.f.u. ratio* | Particle/p.f.u. ratio [symbol] |
|---|---|---|
| EHV-1 | 101.7/1 | 63.8/1 |
| ED71 | 1440/1 | 2128/1 |
| Re71 | 103.5/1 | 107.7/1 |

*Virus from 4 × 10⁸ BHK-21/C13 cells infected with virus at an m.o.i. of 0.01 p.f.u. per cell and harvested at 72 h.p.i. [symbol]
Purified virions from 4 × 10⁸ BHK-21/C13 cells infected with virus at an m.o.i. of 5 p.f.u./cell and harvested at 20 h.p.i.
Suitable gene 71 deletion mutant viruses of the invention were prepared according to the teaching of Sun Y. and Brown S. M. (supra).

### EXAMPLE 1

Briefly, to clone the fragment which contains the gene 71, equine dermal cells (NBL-6) were infected with EHV-1 strain Ab4 (Gibson J. S. et al. Arch. Virol. 124 pp. 351-366 (1992)) at 0.1 pfu/cell and the progeny virions were purified by centrifugation on 5-55% (w/v) sucrose gradients as described by Dumas et al. J. Gen. Virol. 47 pp. 233-235 (1980)). EHV-1 Ab4 genomic DNA was extracted from the purified virions and digested with a range of restriction enzymes. A relevant fragment, for example, the 5.8-kb BamHI/EcoRI fragment (residues 126,517 to 132,305) was cloned into the vector pUC19 so that a plasmid, pU71 containing the 5.8-kb BamHI/EcoRI fragment inserted at BamHI/EcoRI sites, was constructed (FIG. 1). To construct a deletion plasmid, the cloned plasmid was digested by restriction enzymes which cut at unique sites to remove most of the coding sequence of gene 71. The flanking sequences were religated with complementary synthetic oligonucleotides containing an unique Spel site to allow insertion of the lacZ gene and an upstream in-frame stop codon to prevent synthesis of a lacZ fusion protein. The lacZ gene on a 4.1-kb Xbal fragment from pFJ3 (Rixon F. J. and McLauchlan J., J. Gen Virol. 71 pp. 2931-2939 (1990)) was inserted into the Spel site. lacZ was in the same orientation as the gene transcript. The construct could encode only a very short polypeptide of the remaining N-terminal amino acids of the deleted gene. In this way, deletion plasmid pD71 with a deletion from the Mscl to the SgraAl site (residues 129,211-131,022) in pU71, was generated (FIG. 1).

### EXAMPLE 2

For generation of virus mutants, 1-2 μg of EHV-1 Ab4 DNA was cotransfected into BHK21/C13 cells (MacPherson I. and Stoker M. G. Virology 16 pp. 147-151 (1962)) with varying amounts of the linearized deletion plasmid pD71, (0.2 to 4 μg, an approximately 2- to 20-fold molar excess) in the presence of carrier calf thymus DNA using the calcium phosphate precipitation/DMSO method described by Stow N. D., and Wilkie N. M., J. Gen. Virol. 33 pp. 447-458 (1976). The cells were incubated at 37° in Eagle's medium containing 5% newborn calf serum. When the c.p.e. was widespread, the virus was harvested and titrated on BHK21/C13 cells under methylcellulose. Two days after the infection, a further 2 ml of methylcellulose medium containing 0.7 mg/ml X-gal was added to each plate. Individual blue plaques were isolated for further rounds of plaque purification. A mutant with a lacZ substitution was isolated: ED71 with a deletion of 1811 bp from the 2393 bp gene 71 ORF. The deleted region of the mutant was confirmed by Southern blotting with a probe of the ³²P-labelled deleted sequence. The structure of the virus mutant was confirmed by Southern blotting and restriction enzyme digestion of ³²P-labelled viral DNA prior to the preparation of virus stock. The restriction enzyme digestion of ³²P-labelled viral DNA is represented diagrammatically in FIG. 2. Gene 71 lies within the 3.8-kb SmaI fragment of wild-type viral DNA. Deletion of gene 71 and substitution by the lacZ gene resulted in the loss of the 3.8-kb fragment and the generation of a new larger fragment of 6.2-kb. The mutant had the expected genome structure, with no other detectable differences from wild-type viral DNA.

### EXAMPLE 3

Growth characteristics of the mutant in tissue culture was also investigated. Monolayers of BHK21/C13 cells were separately infected at a multiplicity of infection (m.o.i.) of 5 pfu/cell and 0.01 pfu/cell with wild-type virus and the deletion and substitution mutant ED71. The culture was harvested and virus was released by sonication at intervals throughout a 72 hour period. Virus titers were measured by plaque assay and the growth patterns were compared with those of wild-type virus. The plaque morphology of the mutants was not obviously different from the wild-type virus plaques.

Mutant ED71 grew more slowly and the final yield was reduced by about 5-fold compared with that of wild-type virus. Similar results were seen at high multiplicity (data not shown), although the reduction in the yield of the ED71 mutant was less than that at low multiplicity. To determine whether the mutant was temperature sensitive or had a host-range phenotype, they were grown at a high m.o.i. of 5 pfu/cell in BHK21/C13 cells at different temperatures (310, 370, and 38.5° C.) and at 37° C. in NBL-6, Vero, HFL, and 3T6 cells. The cultures were harvested at 24 hour post-infection and progeny virus was titrated in BHK21/C13 cells. The ED71 mutant at 38° C. grew 10-fold less well than at 31° and compared to wild-type virus at 38.5° C. (data not shown). The slightly impaired growth of the ED71 mutant was apparent in NBL-6, Vero, HFL, and 3T6 as well as in BHK21/C13 cells. Thus it is concluded that gene 71 is nonessential for EHV-1 growth in cell culture.

### EXAMPLE 4

### Infection Experiments: Mortality and Clinical Signs

### Materials and Methods

Virus Strains

Wild-type and mutant viruses were grown either in RK cells at the Department of Clinical Veterinary Medicine, Cambridge or in BHK cells at the Institute of Virology, Glasgow. Wild-type for primary infection experiments was EHV-1 strain Ab4p. Virus used to challenge previously immunised mice was EHV-l strain Ab4.

Mouse Model

Female Balb/c mice were obtained at 3-4 weeks of age (Bantin and Kingman, UK). Mice were inoculated intranasally under isofluorane/oxygen anaesthesia.

Tissue Culture

RK cell monolayers were cultured in Eagle's Minimum Essential Medium (EMEM) with Earle's Salts with 10% newborn calf serum.

Virus Titration

Tissue samples obtained from three mice per group were homogenised using an Ultraturrax motorised homogeniser. Samples were then sonicated in an ice-cold waterbath and centrifuged at low speed to separate cellular debris. Ten-fold serial dilutions of the supernatant were made and 100 μl of each dilution inoculated onto confluent monolayers of RK cells, in duplicate. Virus was allowed to adsorb to the cell sheet for 45 minutes before all samples were overlayed with medium containing 4% foetal calf serum and 2% carboxymethylcellulose. Plates were incubated at 37° C. for about 3 days and then washed in sterile phosphate buffered saline prior to fixing and staining with crystal violet in 20% ethanol.

Experimental Protocol

At days 1, 3 and 5 post-infection groups of three mice were euthanased with 0.15 ml of pentobarbitone sodium (Sagatal, Rhône Merieux), tissues removed, placed in 1 ml of virus isolation medium, frozen at −70° C. and then titrated for virus growth. Tissue samples taken were lung, turbinates, olfactory bulb and trigeminal ganglia. Clinical signs were monitored in a separate group of mice from day 0 to day 8 post-infection. Blood samples were taken on days 8, 16, 23 and 30 post-infection for immunological tests. A group of surviving animals were then challenged with a dose of 5×10⁶ pfu/mouse of EHV-1 strain Ab4. Tissue samples were taken as above and clinical signs monitored. Mortality and clinical results are shown in Table 2. Virus titre results are shown in FIGS. 3 to 8 and Tables 4(a)-4(d) inclusive.

### Mortality and Clinical Signs

TABLE 2

| Virus | Mortality | Clinical Signs* |
|---|---|---|
| Ab4p | 77% | Severe |
| ED71 | 8% | Mild |
| ED71 Rev | 60% | Severe |

*Clinical signs observed between Day 1 and Day 8 post-infection.

### EXAMPLE 5

### Immunology —ELISA

The protocol of Tewari D., et al (1994) Journal of Gen. Virol. 75 pp. 1735-1741 was followed. Results are shown in Table 3.

TABLE 3
ELISA

| Acute Phase | | | | |
|---|---|---|---|---|
| Virus | Day 8 p.i. | Day 16 p.i. | Day 23 p.i. | Day 30 p.i. |
| w/t (C.) | 1:25 | 1:125 | 1:125 | 1:125 |
| ED71 | 1:25 | 1:125 | 1:125 | 1:625 |
| Post Challenge | | | | |
| Virus | Day 3 | Day 5 | Day 8 | |
| w/t (C.) | 1:125 | 1:625 | 1:3125 | |
| ED71 | 1:625 | 1:625 | 1:3125 | |

TABLE 4(a)
Day +1 Post Challenge

| | | | No. of +ve | Log₁₀ |
|---|---|---|---|---|
| | MEAN | RANGE | Mice | Reduction MEAN |
| Lung | | | | |
| Negative | 4.3 | 4.6 | 3/3 | — |
| control | | 4.0 | | |
| Positive | 2.6 | 3.3 | 3/3 | 1.7 |
| control | | 2.0 | | |
| Gene | 2.9 | 4.2 | 2/3 | 1.4 |
| Deletion 71 | | <0.7 | | |
| Turbinates | | | | |
| Negative | 4.3 | 4.8 | 3/3 | — |
| control | | 3.9 | | |
| Positive | 3.1 | 3.3 | 3/3 | 1.2 |
| control | | 3.0 | | |
| Gene | 3.0 | 4.3 | 2/3 | 1.3 |
| Deletion 71 | | <0.7 | | |
| Olfactorybulb | | | | |
| Negative | 2.3 | 2.5 | 3/3 | — |
| Control | | 2.0 | | |
| Positive | 1.3 | 1.6 | 3/3 | 1.0 |
| Control | | 1.0 | | |
| Gene | 2.0 | 2.2 | 3/3 | 0.3 |
| Deletion 71 | | 1.7 | | |

TABLE 4(b)
Day +3 Post Challenge

| | | | No. of +ve | Log₁₀ |
|---|---|---|---|---|
| | MEAN | RANGE | Mice | Reduction MEAN |
| Lung | | | | |
| Negative | 4.9 | 5.2 | 3/3 | — |
| Control | | 4.6 | | |
| Positive | 0.9 | 1.0 | 2/3 | 4.0 |
| Control | | <0.7 | | |
| Gene | 0.8 | 0.9 | 1/3 | 4.1 |
| Deletion 71 | | <0.7 | | |
| Turbinates | | | | |
| Negative | 4.6 | 5.2 | 3/3 | — |
| Control | | 4.1 | | |
| Positive | <0.7 | <0.7 | 0/3 | >3.9 |
| Control | | <0.7 | | |
| Gene | <0.7 | <0.7 | 0/3 | >3.9 |
| Deletion 71 | | <0.7 | | |
| Olfactorybulb | | | | |
| Negative | 1.8 | 2.2 | 3/3 | — |
| Control | | 1.4 | | |
| Positive | 0.8 | 0.9 | 1/3 | 1.0 |
| Control | | <0.7 | | |
| Gene | 0.8 | 0.9 | 1/3 | 1.0 |
| Deletion 71 | | <0.7 | | |

TABLE 4(c)
Day +5 Post Challenge

| | | | No. of +ve | Log₁₀ |
|---|---|---|---|---|
| | MEAN | RANGE | Mice | Reduction MEAN |
| Lung | | | | |
| Negative | <0.7 | <0.7 | 0/3 | — |
| Control | | <0.7 | | |
| Positive | <0.7 | <0.7 | 0/3 | — |
| Control | | <0.7 | | |
| Gene | <0.7 | <0.7 | 0/3 | — |
| Deletion 71 | | <0.7 | | |
| Turbinates | | | | |
| Negative | 2.9 | 5.2 | 3/3 | — |
| Control | | 4.1 | | |
| Positive | <0.7 | <0.7 | 0/3 | >2.2 |
| Control | | <0.7 | | |
| Gene | <0.7 | <0.7 | 0/3 | >2.2 |
| Deletion 71 | | <0.7 | | |
| Olfactorybulb | | | | |
| Negative | 0.75 | 2.2 | 1/3 | — |
| Control | | 1.4 | | |
| Positive | <0.7 | 0.9 | 0/3 | <0.05 |
| Control | | <0.7 | | |
| Gene | <0.7 | 0.9 | 0/3 | <0.05 |
| Deletion 71 | | <0.7 | | |

TABLE 4(d)
Day +8 Post Challenge

| | | | No. of +ve | Log₁₀ |
|---|---|---|---|---|
| | MEAN | RANGE | Mice | Reduction MEAN |
| Lung | | | | |
| Negative | <0.7 | <0.7 | 0/3 | — |
| Control | | <0.7 | | |
| Positive | <0.7 | <0.7 | 0/3 | — |
| Control | | <0.7 | | |
| Gene | <0.7 | <0.7 | 0/3 | — |
| Deletion 71 | | <0.7 | | |
| Turbinates | | | | |
| Negative | <0.7 | <0.7 | 0/3 | — |
| Control | | <0.7 | | |
| Positive | <0.7 | <0.7 | 0/3 | — |
| Control | | <0.7 | | |
| Gene | <0.7 | <0.7 | 0/3 | — |
| Deletion 71 | | <0.7 | | |
| Olfactorybulb | | | | |
| Negative | <0.7 | <0.7 | 0/3 | — |
| Control | | <0.7 | | |
| Positive | <0.7 | <0.7 | 0/3 | — |
| Control | | <0.7 | | |
| Gene | <0.7 | <0.7 | 0/3 | — |
| Deletion 71 | | <0.7 | | |

### EXAMPLES SECTION 2

1. Experimental details

The trial was performed in pony colts using 3 animals per group and two groups, one vaccinated and one not (control group). The trial animals were selected on the basis that they had no or low EHV-1 neutralising and EHV-1 complement fixing (CF) antibodies. The experimental groups were kept in separate rooms in isolation with filtered air in and out. Colts, 7, 15 and 20 were each vaccinated intranasally with 6.0 log10 TCID₅₀ of gene 71 deleted EHV-1 (ED71), in 2.0 mls of MEM (Gibco) containing neomycin (100 μg/ml), 2% γ-irradiated foetal calf serum (FCS) (Tissue Culture Services), giving 1.0 ml into each nostril. Following vaccination both vaccinated test colts (internal numbering 7, 15 and 20) and control colts (internal numbering 5, 8 and 16) were tested for virus replication in the upper respiratory tract by taking nasal swabs daily for 2 weeks. All six animals were bled at intervals and their sera tested for EHV-1 neutralising and CF antibodies (Table 7). Intranasal challenge infection with wild type strain AB-4 was conducted 51 days after vaccination when colts in both groups were each given 6.0 log₁₀ TCID₅₀ of AB-4, in 2.0 mls of MEM medium supplemented with 2% FCS. Following challenge the procedures performed were the same as those after vaccination, i.e. assessment of virus growth in the upper respiratory tract (Table 5).

TABLE 5
Experimental groups and procedures

| | Colt | | Procedures after | Challenge & |
|---|---|---|---|---|
| Group | No | Vaccination | vaccination | Procedures |
| Test | 7, 15, | Intranasally | All 6 colts | All 6 colts |
| Group | 20 | 6.0 log₁₀ of | (i) Nasal swabs | (i) Nasal swabs |
| | | ED71 in 2.0 mls | day 1-14 | day 1-14 |
| | | | | Intranasally |
| | | | | with 6.0 log₁₀ |
| | | | | TCID₅₀ of wild |
| | | | | type strain AB-4 |
| | | | | and |
| | | | | (ii) Leukocyte |
| | | | | viraemia on |
| | | | | days 0, 1, 3, |
| | | | | 5, 7, 9, 11 & |
| | | | | 13. |
| Control | 5, 8, | None (control) | | |
| Group | 16 | | | |

2. Results

2.1 Replication of ED71 virus in the upper respiratory tract

Viruses were isolated from nasal swabs in MEM medium supplemented as described above, following standard procedures. Results of virus isolation from daily nasal swabs following intranasal vaccination are given in Table 6. ED71 virus at low titre (mostly below 3.0 log₁₀ TCID₅₀/ml) was isolated from 2 of 3 vaccinated colts, on days 2 and 3 from colt 7, and days 1 to 5 from colt 15. No EHV-1 was recovered from control colts from daily nasal swab samples over 14 days.

2.2 Serological responses following vaccination

Sera were titrated fro virus neutralising (VN) and complement fixing (CF) antibodies. Results of VN tests performed according to the method of Thompson G. R., et al Equine Vet. Journal Vol. 8 pp 58-65, for both post vaccination and challenge are given in Table 7 and those for CF test performed according to the method of Thompson et al supra (vaccination only) are given in Table 8.

In the VN test against two different strains of EHV-1 namely ED71 virus (parent strain AB-4) and M8 no significant differences in titres were recorded. In the vaccinated group all three colts were just detectably VN antibody positive at intranasal vaccination. All three animals responded with significant (≧4-fold rise) antibody response, Nos 7 & 20 by week four and No 15 by week two.

There was no VN antibody rise in the control animals until after challenge. By the CF test against EHV-1 two (15 and 20) of three colts showed a significant rise (≧4-fold rise) by week two after vaccination; colt No 7 had high activity at vaccination (Table 8). Control animals (5, 8 and 16) did not show significant change in CF antibody titres.

In keeping with the virus isolation results, there was no seroconversion in control animals indicating the absence of a field infection or EHV-1 recrudescence.

3. Challenge findings

3.1 Challenge virus replication in the upper respiratory tract

Virus isolation results from nasal swabs are given in Table 9. Virus at low titre (2.0 log₁₀ TCID₅₀/ml) isolated from only one (no 7) of three colts on two occasions (day 1 and 2). This was in marked contrast to the control colts (5, 8 and 16) from which virus was recovered for 3 (no 5) to 5 to 6 days (Nos 8 and 16) at much higher titres.

3.2 Viraemia due to the challenge virus

Challenge EHV-1 isolation from leukocytes is given in Table 10. There was no challenge virus detected in ED71 vaccinated colts. In contrast all three control colts became viraemic yielding, at peak between 12 to 200 infected leukocytes/2×10⁷ cells.

TABLE 6
Vaccine virus replication in upper respiratory tract

| | | Virus isolated (log₁₀TCID₅₀/ml) from nasal swabs | | | | | | | | | | | | | | |
|---|---|---|---|---|---|---|---|---|---|---|---|---|---|---|---|---|
| | Colt | following intranasal vaccination (days) | | | | | | | | | | | | | | |
| Group | No | 0 | 1 | 2 | 3 | 4 | 5 | 6 | 7 | 8 | 9 | 10 | 11 | 12 | 13 | 14 |
| TEST | 7 | —^{a} | 2.7 | 1.5 | — | — | — | — | — | — | — | — | — | — | — | — |
| (ED71) | 15 | — | 1.5 | 4.4 | 1.7 | 2.0 | 1.5 | — | — | — | — | — | — | — | — | — |
| | 20 | — | — | — | — | — | — | — | — | — | — | — | — | — | — | — |
| CONTROL | 5 | — | — | — | — | — | — | — | — | — | — | — | — | — | — | — |
| | 8 | — | — | — | — | — | — | — | — | — | — | — | — | — | — | — |
| | 16 | — | — | — | — | — | — | — | — | — | — | — | — | — | — | — |

^{a}denotes no EHV-1 isolated in equine dermal cells from 4 × 200μ1 of the lowest (10⁻¹) dilution of the nasal swab titration.

TABLE 7
Virus neutralising (VN) antibody responses

| | | Circulating VN and EHV-M8antibody^{a} to EHV-1-ED71 | | | | | |
|---|---|---|---|---|---|---|---|
| | Colt | | week 0 | | | week +7 | |
| Group | No | week −1 | vac^{b} | week +2 | week +4 | challenge^{c} | week +10 |
| TEST | 7 | 8, 8 | 4, 8 | 16, 16 | 32, 32 | 16, 32 | 32, 32 |
| | 15 | 4, 4 | 4, 8 | 32, 64 | 64, 64 | 64, 64 | 64, 128 |
| | 20 | 8, 8 | 8, 8 | 16, 16 | 32, 64 | 32, 32 | 32, 32 |
| CONTROL | 5 | 4, 4 | 4, 4 | 4, 4 | 4, 4 | 4, 4 | 32, 32 |
| | 8 | <4, <4 | <4, <4 | <4, <4 | <4, <4 | <4, <4 | 32, 32 |
| | 16 | 4, 4 | 4, 4 | <4, 4 | 4, 4 | 4, 4 | 32, 64 |
| ^{a}VN tests were performed against ED71 virus (lefthand figures) and EHV-1 M8 (righthand figures). Titres denote reciprocal of | | | | | | | |
| #serum dilution completely neutralising. 200 (ED71) to 316 (M8) TCID₅₀ of EHV-1. | | | | | | | |

^{b}Vac denotes vaccination on week 0.
^{c}Challenge on week +7.

TABLE 8
Complement fixing (CF) antibody responses to EHV-1

| | | Circulating CF antibody to EHV-1 (AB-4) | | | |
|---|---|---|---|---|---|
| | Colt | | week 0 | | |
| Group | No | week −1 | vac^{d} | week +2 | week 1 +4 |
| TEST | 7 | 160 | 320 | 640 | 640 |
| | 15 | 40 | 10 | 640 | 640 |
| | 20 | 40 | 20 | 320 | 640 |
| CONTROL | 5 | 20 | 5 | 0 | 0 |
| | 8 | 10 | 5 | 5 | 5 |
| | 16 | 20 | 40 | 40 | 40 |

^{a}vac denotes vaccination on week 0.

TABLE 9
Challenge virus replication in the upper respiratory tract

| | | Virus isolated (log₁₀TCID₅₀/ml) from nasal swabs | | | | | | | | | | | | | |
|---|---|---|---|---|---|---|---|---|---|---|---|---|---|---|---|
| | Colt | following intranasal challenge (days) | | | | | | | | | | | | | |
| Group | No | 0 | 1 | 2 | 3 | 4 | 5 | 6 | 7 | 8 | 9 | 10 | 11 | 12 | 13 |
| TEST | 7 | —^{a} | — | 2.0 | 2.0 | — | — | — | — | — | — | — | — | — | — |
| (ED71) | 15 | — | — | — | — | — | — | — | — | — | — | — | — | — | — |
| | 20 | — | — | — | — | — | — | — | — | — | — | — | — | — | — |
| CONTROL | 5 | — | 3.7 | 2.0 | — | — | 2.7 | — | — | — | — | — | — | — | — |
| | 8 | — | 2.7 | 3.7 | 3.5 | 2.5 | 3.7 | 2.0 | — | — | — | — | — | — | — |
| | 16 | — | 4.5 | 4.3 | 3.4 | 3.7 | 2.5 | — | — | — | — | — | — | — | — |

TABLE 10
Leukocyte viraemia following intranasal EHV-1 challenge

| | | Number of ED71 virus infected leukocytes*/ | | | | | | | |
|---|---|---|---|---|---|---|---|---|---|
| | Colt | 2 × 10⁷cells days after challenge | | | | | | | |
| Group | No | 0 | 1 | 3 | 5 | 7 | 9 | 11 | 13 |
| TEST | 7 | —^{b} | — | — | — | — | — | — | — |
| (ED71) | 15 | — | — | — | — | — | — | — | — |
| | 20 | — | — | — | — | — | — | — | — |
| CONTROL | 5 | — | — | — | 12 | 5 | 2.5 | — | — |
| | 8 | — | — | — | 200 | 1.3 | — | — | — |
| | 16 | — | — | — | 20 | 2.5 | 2.5 | — | — |
| ^{a}Buffy coat cell layer from 10mls of citrated blood was separated on a percoll cushion washed in phosphate buffered saline and titrated in monolayers of equine dermal cells (Animal Health Trust, Newmarket, Suffolk) using 8 | | | | | | | | | |
| #monolayers/dilution and 3 tenfold dilutions (MEM, 10% γ-irradiated FCS supplemented with neomycin). | | | | | | | | | |

^{b}No EHV-1 isolated after two serial passages at 37° C.

1. Vaccine formulation comprising a live, recombinant EHV-1 virus modified so as to contain a dysfunctional gene 71 region located within the U_{S} region of the virus genome and a pharmaceutically acceptable carrier.

2. A vaccine formulation according to claim 1 comprising a live, recombinant, attenuated immunogenic EHV-1 gene 71 deletion mutant virus and a pharmaceutically acceptable carrier.

3. A vaccine formulation according to claim 1 or claim 2 wherein the dysfunctional gene 71 region of the recombinant EHV-1 virus comprises a deletion of at least one nucleotide between nucleotide 129,096 and nucleotide 131,489 of a wild type EHV-1 genome.

4. A vaccine formulation according to any one of claims 1 to 3 wherein the recombinant EHV-1 comprises a marker gene.

5. A live, recombinant EHV-1 comprising a dysfunctional gene 71 region for use as a vaccinating agent.

6. A live, recombinant, attenuated immunogenic EHV-1 gene 71 deletion mutant virus for use as a vaccinating agent.

7. Use of a live, recombinant, EHV-1 gene 71 deletion mutant virus in the manufacture of an EHV-1 vaccine for the prophylaxis and/or therapy of EHV-1 infection.

8. A method of treating an animal which comprises administering to an animal a vaccine composition comprising a live, recombinant EHV-1 virus modified so as to contain a dysfunctional gene 71 region located within the Us region of the virus genome.

9. A method according to claim 8 wherein the animal is a horse.

10. A method according to claim 8 or claim 9 wherein the vaccine composition comprises a recombinant, live, attenuated, immunogenic EHV-1 gene 71 deletion mutant virus.
